(19) United States
(12) Patent Application Publication
(10) Pub. No.: US 2020/0318054 A1
(43) Pub. Date: Oct. 8, 2020

(54) AIR-STIRRED TANK REACTOR (ASTR) FOR PRODUCTION OF MICROORGANISMS AND CELL CULTURES

(71) Applicant: ARIZONA BOARD OF REGENTS ON BEHALF OF THE UNIVERSITY OF ARIZONA, Tucson, AZ (US)

(72) Inventors: Joel L. Cuello, Tucson, AZ (US); Jack Welchert, Tucson, AZ (US); Andres P. Mayol, Tucson, AZ (US); Mark Jendrisak, Tucson, AZ (US)

(21) Appl. No.: 16/908,515

(22) Filed: Jun. 22, 2020

Related U.S. Application Data
(63) Continuation-in-part of application No. PCT/US18/67125, filed on Dec. 21, 2018.
(60) Provisional application No. 62/609,675, filed on Dec. 22, 2017.

(51) Int. Cl. C12M 1/00; C12N 5/00; C12M 1/04; B01F 13/02; B01F 7/00; B01F 7/22

(52) U.S. Cl. CPC C12M 29/20; C12N 5/0018; C12M 27/04; C12M 29/06; C12M 29/26; C12M 41/06; B01F 2215/0073; B01F 7/0035; B01F 7/00633; B01F 7/007; B01F 7/00708; B01F 7/225; B01F 13/0216

(57) ABSTRACT

An air-stirred tank reactor (ASTR) and methods of use thereof are described herein. The ASTR is equipped with an impeller or set of impellers that mechanically mixes a liquid culture, as well as sparges gas into the liquid medium. The impeller can further have lighting sources that can illuminate the liquid culture. Unlike conventional bioreactors, the ASTR provides superior liquid mixing, efficient gas mass transfer, and a low-shear culture environment through appropriate impeller rotational speed and sparging rate.

### CROSS-REFERENCES TO RELATED APPLICATIONS

This application is a continuation-in-part and claims benefit of PCT/US18/67125, filed Dec. 21, 2018, which claims priority to U.S. Provisional Application No. 62/609,675, filed Dec. 22, 2017, the specification(s) of which is/are incorporated herein in their entirety by reference.

### BACKGROUND OF THE INVENTION

### Field of the Invention

The present invention relates to bioreactors, namely, an air-stirred tank reactor (ASTR) for cultivating microorganisms and cell cultures used in applications such as biotechnology, pharmaceutical, and food industries.

The photoautotrophic growth of microorganisms or cells is enabled by the photosynthetic capacity of the chlorophyll-containing microorganisms or cells, whereby carbon dioxide (CO₂), through photosynthetic carbon fixation, serves as the carbon (or food) source. Photoautotrophic growth requires the presence of light for photosynthesis to occur. A steady supply of CO₂ when light is available also promotes culture growth.

By contrast, heterotrophic growth takes place when the microorganisms or cells, in the absence of photosynthetic CO₂ fixation, rely on exogenous carbon-based molecules, typically sugars such as glucose or sucrose, present in the liquid culture medium as their carbon (or food) source. Heterotrophic growth necessitates a sterile or axenic growth environment to avoid culture contamination; otherwise, unwanted and competing bacteria and other microorganism would grow in the culture owing to the presence of the carbon-based food source. This mode of growth also requires a steady supply of oxygen (O₂) which the microorganisms or cells need as they breakdown the carbon-based molecules through the process of respiration. Since light is not essential, heterotrophic production is generally carried out in darkness. Mixotrophic growth takes place when the microorganisms or cells grow both photoautotrophically and heterotrophically.

### Description of Related Art Including Information Disclosed

Oxygen delivery into the liquid culture is critically important for heterotrophic and mixotrophic production, while CO₂ delivery is critically important for photoautotrophic and mixotrophic production. And yet gas delivery into a bioreactor to achieve uniform distribution of adequate levels of dissolved gas throughout the volume of a scalable bioreactor remains a significant challenge. This is the principal problem that the present invention addresses and does so successfully.

The present invention features an air-stirred tank reactor (ASTR) as a bioreactor that could be used for the heterotrophic, mixotrophic, and photoautotrophic growth and production of microorganisms (bacteria, fungi, algae, etc.) as well as cell cultures of plants, animals, insects, and others. The ASTR features the following advantages over conventional reactors: 1) superior liquid mixing; 2) efficient gas mass transfer; 3) well-mixed distribution of dissolved gas at desired levels in the liquid medium; 4) regulated or low-shear culture environment as desired; and 5) effective internal lighting within the bioreactor as desired.

### BRIEF SUMMARY OF THE INVENTION

It is an objective of the present invention to provide systems and methods that allow for simultaneous mixing of a liquid and sparging of gas into the liquid using a single mechanism (e.g., a perforated impeller) providing a synergistic effect on hydrodynamic mixing and gas transfer for optimal growth and production of microorganisms and cell culture, as specified in the independent claims. Embodiments of the invention are given in the dependent claims. Embodiments of the present invention can be freely combined with each other if they are not mutually exclusive.

In some aspects, the present invention features an air-stirred tank reactor (ASTR) equipped with an impeller or set of impellers that not only mechanically mixes the liquid culture, but also sparges gas into the liquid medium. In one aspect, gas is introduced into the liquid medium through the impeller's perforated surfaces. In another aspect, gas is introduced into the liquid medium by using sparger tapes attached to the impeller surfaces. In some embodiments, the impeller of the ASTR may be any type of conventional impeller, designed to have the advantages of providing superior liquid mixing, efficient gas mass transfer, and a low-shear culture environment through appropriate impeller rotational speed and sparging rate.

In some embodiments, the ASTR can transmit internal lighting into the liquid culture through light sources (e.g., light emitting diodes) that are either embedded on the impeller surfaces or the use of light tapes attached to the impeller surfaces. In other embodiments, the ASTR may utilize a single impeller or multiple impellers. In further embodiments, the ASTR impellers may be constructed from metal, ceramic, transparent or non-transparent polymer or other material.

One of the unique and inventive technical features of the present invention is the use of porous impellers as a means of introducing bubbles through the propeller. Without wishing to limit the invention to any theory or mechanism, this technical feature of the ASTR makes it unique compared to conventional continuous stirred tank reactors (CSTR) by allowing a single mechanism (e.g., porous or perforated impellers) to effect both liquid mixing and aeration (e.g., gas sparging, gas transfer) functions. Conventional bioreactors typically have a separate liquid mixing mechanism (e.g., impeller) and a separate aeration mechanism (e.g., sparger ring). The ASTR of the present invention allows for creation of gas bubbles from the pores of an impeller for transfer of gas, such as oxygen and carbon dioxide, into the liquid, and also for hydrodynamic mixing of the liquid. The combined action of the rotating impeller and the impeller-originating bubbles results in a synergistic improvement on the liquid mixing and on the gas transfer of oxygen into the liquid, resulting in enhanced growth and production of the culture. Additional advantages of the ATSR compared to the CSTR include: (1) mixing of the liquid medium and sparging gas into the liquid culture simultaneously, while rotating or at rest, through either the perforated surfaces of the impeller(s) or the use of sparger tapes attached to the impeller surfaces; (2) generating novel hydrodynamic mixing patterns within the reactor; and (3) enabling the use of any design type or geometric configuration, including conventional impellers such as a flat blade turbine (Rushton impeller), a spiral turbine, a propeller, a pitched blade turbine, a helical ribbon, a helical screw, a helical ribbon screw, etc. In further embodiments, the ASTR impellers may also transmit internal lighting into the liquid culture as desired through light sources (e.g., light emitting diodes) that are either embedded on the impeller surfaces or the use of light tapes attached to the impeller surfaces. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a non-limiting embodiment of an air stirred tank reactor (ASTR) whose impeller, in contrast to that of a conventional continuous stirred tank reactor (CSTR), can mechanically mix the liquid culture as well as sparge gas into the liquid medium through the impeller's perforated surfaces. FIG. 1B shows a non-limiting embodiment of an ASTR with two impellers.

FIG. 2A shows a conventional CSTR whose impeller simply mixes the liquid medium. FIG. 2B shows a conventional gas sparger ring.

FIGS. 3A-3C show various embodiments of conventional impellers that may be used in accordance with the ASTR of the present invention. FIG. 3A shows non-limiting examples of radial flow impellers, including a Flat Blade Turbine or Rushton Impeller (left side) and a Sweptback or Curved Turbine (Spiral Turbine; right side) FIG. 3B shows non-limiting examples of axial flow impellers, including a propeller (left side) and 45° Pitch Blade Turbine (right side). FIG. 3C shows non-limiting examples of laminar flow impellers including Helical Ribbon Impeller (top left), Helical Screw Impeller (top right), Helical Ribbon Screw Impeller (bottom left), and an Anchor Screw (bottom right) (Fogler & Gurman, 2008, Mixing in Chemical Reactors, University of Michigan).

FIGS. 4A-4B show schematics of hydrodynamic flow or mixing patterns for a radial flow impeller (FIG. 4A) and an axial flow impeller (FIG. 4B) (Fogler & Gurman, 2008, Mixing in Chemical Reactors, University of Michigan).

FIGS. 5A-5D show non-limiting embodiments of the ASTR, showing different types of impeller, which may be single or multiple within a bioreactor.

FIGS. 6A-6B show non-limiting embodiments of the ASTR having sparger tapes attached to impellers' surfaces.

FIGS. 7A-7B show non-limiting embodiments of the ASTR having light sources disposed on the impeller surfaces, or light tapes attached to the impeller surfaces.

FIGS. 7C-7D show non-limiting embodiments of the light sources (dotted lines) embedded inside the impeller. In some preferred embodiments, the impellers are clear and/or transparent to allow for light transmission.

FIG. 8 shows the Air Volume Fraction results from Computational Fluid Dynamics (CFD) simulations conducted on an ASTR.

FIG. 9 shows the Air Volume Fraction results from CFD simulations conducted on a CSTR.

FIG. 10 shows the Air Velocity results from CFD simulations conducted on an ASTR.

FIG. 11 shows the Air Velocity results from CFD simulations conducted on a CSTR.

FIG. 12 shows the Water Velocity Fraction results from CFD simulations conducted on an ASTR.

FIG. 13 shows the Water Velocity results from CFD simulations conducted on a CSTR.

### DESCRIPTION OF PREFERRED EMBODIMENTS

Following is a list of elements corresponding to a particular element referred to herein:
100 air-stirred tank reactor (ASTR)
105 reactor vessel
110 gas-sparging mixing system
120 impeller
122 impeller surface
125 pores
127 rotatable shaft
128 interior channel of the rotatable shaft
130 gas-delivering channel
140 sparger tape
150 light source

As used herein, the term “synergistic” refers to the interaction or cooperation of two or more organizations, substances, or other agents to produce a combined effect greater than the sum of their separate effects. For example, as it pertains to the present invention, the methods described herein produces a synergistic effect on both liquid mixing and gas transfer through the combined action of the rotating impeller and the impeller-originating or impeller-generated bubbles, which then translates into improved/enhanced growth and production of the culture being grown compared to conventional methods that have separate mechanisms for fluid mixing and gas sparging.

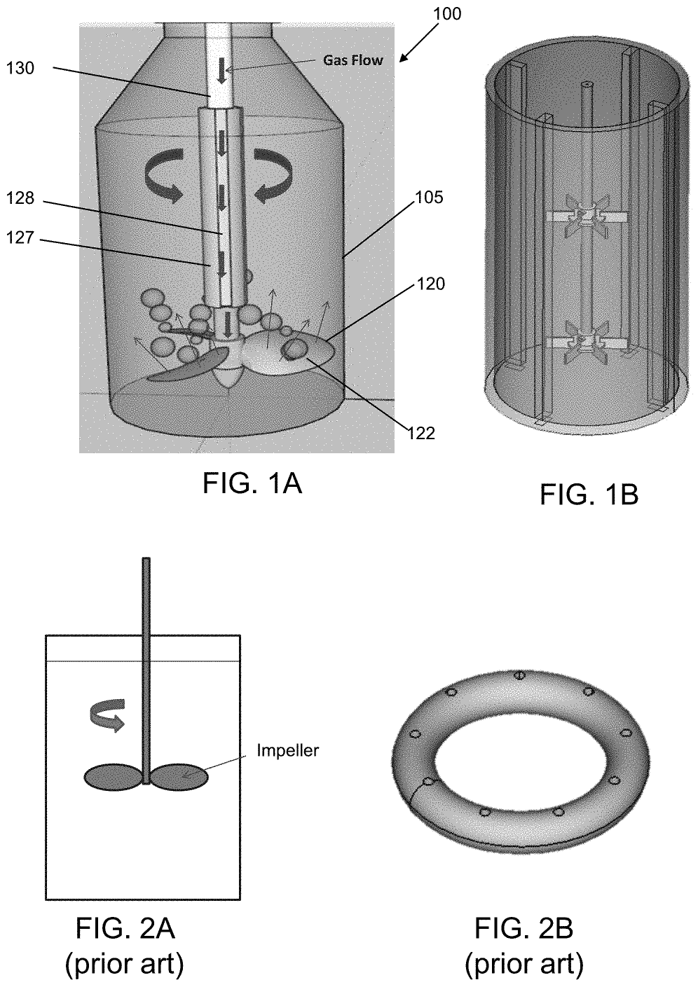

Referring to FIG. 1A, according to some embodiments, the present invention may feature a gas-sparging mixing system (110) comprising at least one impeller (120) for circulating fluids, and a gas-delivering channel (130) fluidly connected to the impeller (120). In some embodiments, the impeller (120) may be operatively connected to a rotatable shaft (127) such that the impeller (120) can to rotate upon axial rotation of the rotatable shaft (127). In some embodiments, the impeller (120) may have pores (125) disposed on a surface (122) of the impeller. In other embodiments, the gas-delivering channel (130) is configured to transport gas through the impeller (120). The gas may then exit through the pores (125) of the impeller so that the fluids being circulated by the impeller (120) is sparged with the gas exiting through the pores (125).

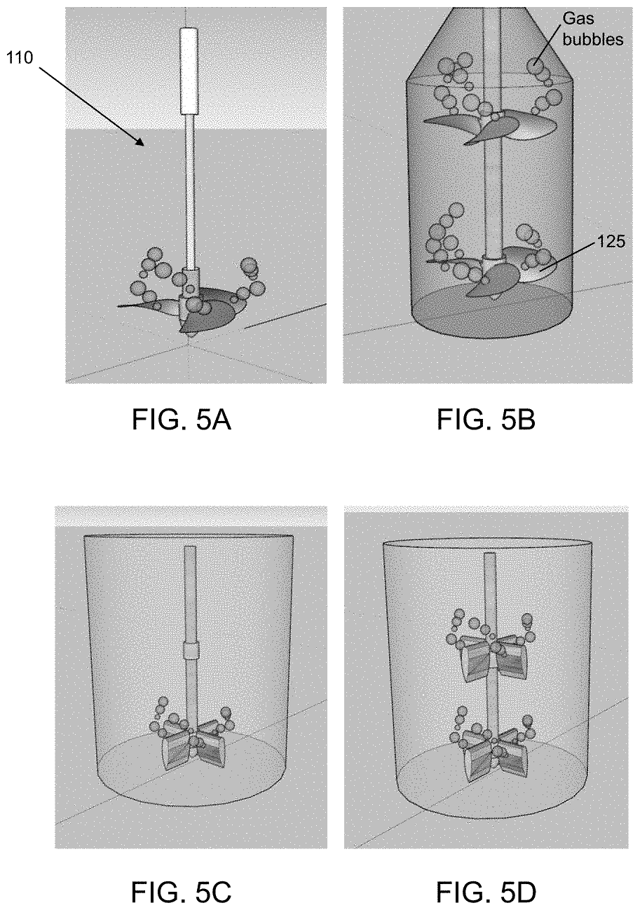

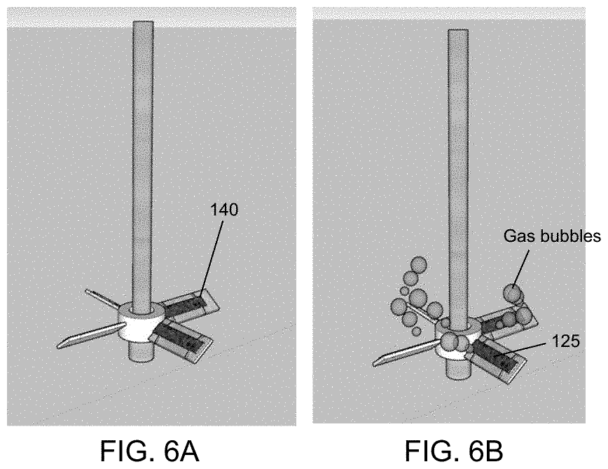

FIG. 1A illustrates how compressed gas may be delivered internally through the impeller with porous surfaces. In some embodiments, gas sparging by the impeller may be implemented in two ways: (1) through the porous or perforated surfaces of the impeller, as shown in FIGS. 5A-5D; or (2) through the use of sparger tapes attached to the impeller surfaces, as shown in FIGS. 6A-6B. In one embodiment, the pores (125) may be embedded on the surface (122) of the impeller. In another embodiment, the pores (125) may be embedded on the sparger tape (140) attached to the surface (122) of the impeller. Gas sparging by the impeller may be implemented while the impeller is rotating and also while the impeller is at rest.

In some embodiments, the gas-delivering channel (130) may comprise tubing that is fluidly connected to the impeller (120). The gas may be transported through the tubes and then exits the impeller via the pores (125). In one embodiment, the tubes of the gas-delivering channel (130) may be disposed through an interior channel (128) of the rotatable shaft. This configuration may be suitable in the case of the pores (125) being embedded directly on the surface (122) of the impeller. For instance, the blades of the impeller may have an input for receiving the gas, which then flows through a hollow interior of the blade, and finally exits through the pores of the blade surface. In another embodiment, the tubes may be disposed paraxial to the rotatable shaft.

In an embodiment where the pores (125) are embedded on the sparger tape (140), the tubes of the gas-delivering channel (130) may be fluidly connected to the sparger tape (140) such that gas is transported through the tubes and exits through the pores (125) of the sparger tape. FIGS. 6A-6B illustrates how sparger tapes may be attached to the impellers' surfaces. In some embodiments, flexible small-diameter tubing, delivering compressed gas into each sparger tape, may be attached to the rotatable shaft (not shown). The use of sparger tapes can enable the use of conventional impellers without the need of using impellers with perforations for gas sparging.

Given that pressurized gas is being conveyed through the sparger pores on the impeller, or on the sparger tape attached to the impeller, the impeller itself may be driven to rotate, partially or exclusively, through the momentum generated on the impeller by the pressure of gas sparging. The gas pressure applied in each blade of the impeller may be set and coordinated so that desired rotational speeds of the impeller may be achieved. This can provide significant savings in energy expenditure by the ASTR bioreactor as compared to that of the conventional CSTR where energy is spent on rotating the impeller independently of the energy spent on sparging gas into the same vessel.

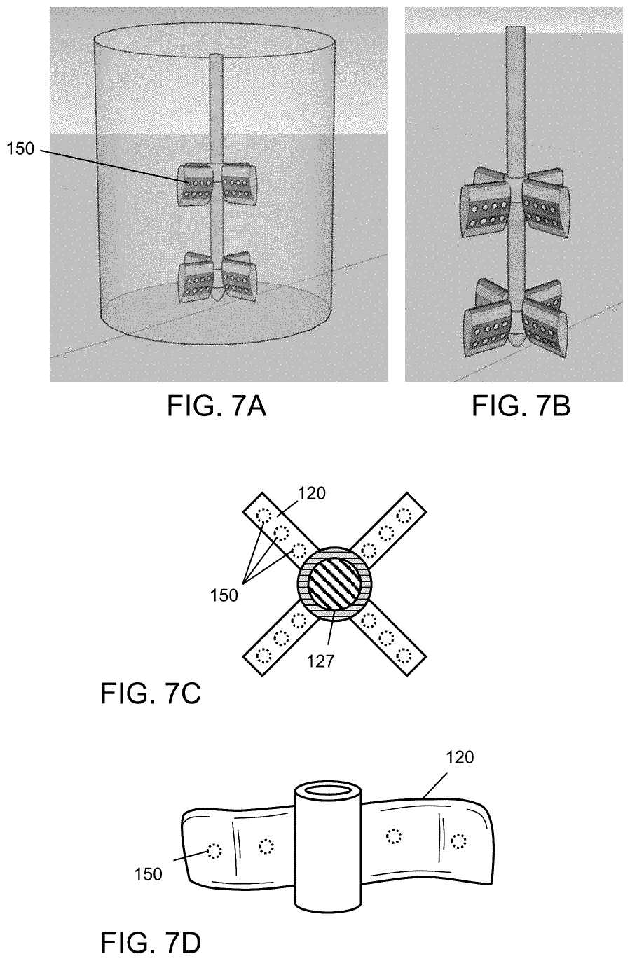

Referring to FIGS. 7A-7B, the impeller of the system (100) may be designed to transmit light to the fluid through the use of light sources. In some embodiments, the impeller may further comprise one or more light sources (150) disposed on the surface (122) of the impeller or embedded within the impeller such that the light source (150) is exposed to the fluids. In one embodiment, the light source (150) may comprise a light emitting diode disposed on the surface (122) of the impeller. In another embodiment, the light source (150) may comprise light tapes attached to the surface (122) of the impeller.

In alternative embodiments, as shown in FIGS. 7C-7D, the light source (150) may be completely embedded or contained within the impeller such that the light source (150) is not directly in contact with the fluids or culture environment. As a non-limiting example, the impeller may comprise a transparent material that allows for light from the embedded light source (150) to transmit through said material. Examples of transparent materials include, but are not limited to, glass, polycarbonate, acrylics, etc.

In some embodiments, as shown in FIGS. 5B, 5D, and 7B, the system (110) may comprise a plurality of impellers (120). The impellers (120) may be arranged parallel to each other so as to form an impeller array or stack. Further still, the impellers (120) may be operatively connected to a single rotatable shaft (127). In alternative embodiments, the impellers (120) may be arranged in series and each operatively connected to its own rotatable shaft. Consequently, each rotatable shaft may have its own gas-delivery tubing fluidly coupled to its respective impeller.

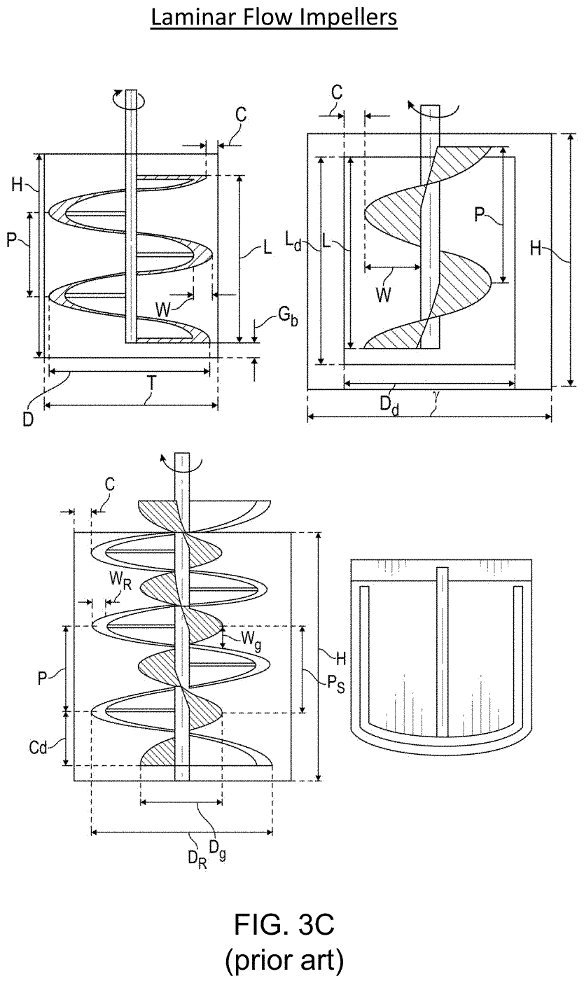

Referring to FIG. 3A-3C, examples of the impeller (120) include, but are not limited to, a flat blade turbine (Rushton impeller), a spiral turbine, a propeller, a pitched blade turbine, a helical ribbon impeller, a helical screw impeller, a helical ribbon screw impeller, and an anchor impeller. In some embodiments, the impeller (120) is constructed from a metal, a ceramic, or a substantially transparent or non-transparent polymer.

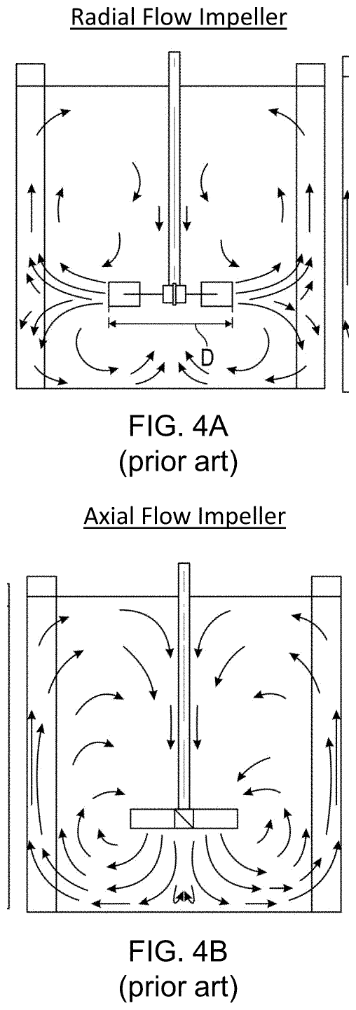

Without wishing to limit the invention to a particular theory or mechanism, the gas-sparging mixing system (110) has the advantages of providing superior liquid mixing, efficient gas mass transfer, and even a low-shear culture environment through appropriate impeller rotational speed and sparging rate. Each type of impeller generates a specific hydrodynamic flow or mixing pattern within the reactor. FIGS. 4A-4B show that hydrodynamic flow or mixing patterns, for instance, for a radial flow impeller and an axial flow impeller. Given that the impeller sparges gas at adjustable flow rates, the system (110) then has the capacity and significant advantage of generating novel hydrodynamic mixing patterns within the reactor to suit the specific mixing needs of particular microbial or cell cultures.

Other independent variables that may be adjusted or modified to effect optimal hydrodynamic flow or mixing patterns within the system include: (1) rotational speed of the impeller(s); (2) pore size on the impeller or sparger tape; (3) pore density on the impeller surface or sparger tape; (4) bubble size generated by the pores on the impeller or sparger tapes; (5) gas flow rate; (6) the application of continuous or intermittent sparging; and (7) frequency and period of intermittent sparging. In some embodiments, the pore size and density on the impeller surface, or on the sparger tape, may be selected to have values such that a gas bubble size generated by the pores can range from nanometers to micrometers, millimeters, centimeters, or greater. For example, an average pore diameter may range from about 1 nm to about 1 μm, or from about 1 μm to about 1 mm, or from about 1 mm up to about 1 cm. In other embodiments, the pore density may range from about 1 pore/cm² to about 100 pores/cm².

Since the present invention provides gas-sparging mixing systems (110) according to the embodiments described herein, it is another objective of the present invention to provide methods of utilizing the system (110). According to some embodiments, the present invention may feature a method of sparging gas into a liquid medium. The method may comprise placing the gas-sparging mixing system (110) in the liquid medium, transporting gas to the impeller (120) via the gas-delivering channel (130), and sparging gas, which exits through the pores (125) of the impeller, into the liquid medium. In other embodiments, the method may further comprise rotating the impeller (120) to cause circulation of the liquid medium.

According to other embodiments, the present invention may feature an air-stirred tank reactor (ASTR) (100) for cultivation of a liquid culture. The ASTR (100) may comprise a reactor vessel (105) configured to contain the liquid culture, and the gas-sparging mixing system (110) disposed in the reactor vessel (105). In one embodiment, the impeller (120) of the gas-sparging mixing system may be configured to circulate the liquid culture. In another embodiment, the gas-delivering channel (130) may be configured to transport gas through the impeller (120), which then exits through the pores (125) and sparges the liquid culture being circulated by the impeller (120).

In some embodiments, the reactor vessel (105) may comprise substantially transparent or non-transparent reactor walls. In other embodiments, the impeller (120) may have a light source (150) disposed on the surface (122) of the impeller for providing effective lighting within the reactor vessel (105). The light source may be exposed to the external culture environment. In alternative embodiments, the light source (150) may be embedded or contained within the impeller such that the light source (150) is not directly in contact with the liquid culture, yet light can be transmitted through the impeller to illuminate the liquid culture.

According to further embodiments, the (ASTR) (100) may be used to cultivate a liquid culture. In one embodiment, the present invention features a method of cultivating the liquid culture contained in a reactor vessel (105). The method may comprise placing the gas-sparging mixing system (110) in the reactor vessel (105) containing the liquid culture, rotating the impeller (120) to cause circulation of the liquid culture in the reactor vessel (105), transporting gas to the impeller (120) via the gas-delivering channel (130), and sparging the liquid culture with the gas that exits through the pores (125) of the impeller. In some embodiments, the method may further comprise illuminating the liquid culture via the light source (150) disposed on the surface (122) of the impeller. Without wishing to limit the invention to a particular theory or mechanism, the ASTR (100) and method of use thereof may provide superior liquid mixing, efficient gas mass transfer, well-mixed distribution of dissolved gas at desired levels in the liquid culture, and a regulated or low-shear culture environment as desired through appropriate impeller rotational speed and sparging rate.

In some embodiments, the ASTR (100) may be used for heterotrophic, mixotrophic, or photoautotrophic growth and production of microorganisms such as bacteria, fungi, or algae, or of cell cultures such as plant, animal, or insect culture, in a liquid medium. In other embodiments, the ASTR (100) may be used in applications such as those in biotechnology, pharmaceutical, or food industries.

### EXAMPLE

The following is a non-limiting example of the present invention. It is to be understood that said example is not intended to limit the present invention in any way. Equivalents or substitutes are within the scope of the present invention.

Computational Fluid Dynamics (CFD) Simulations

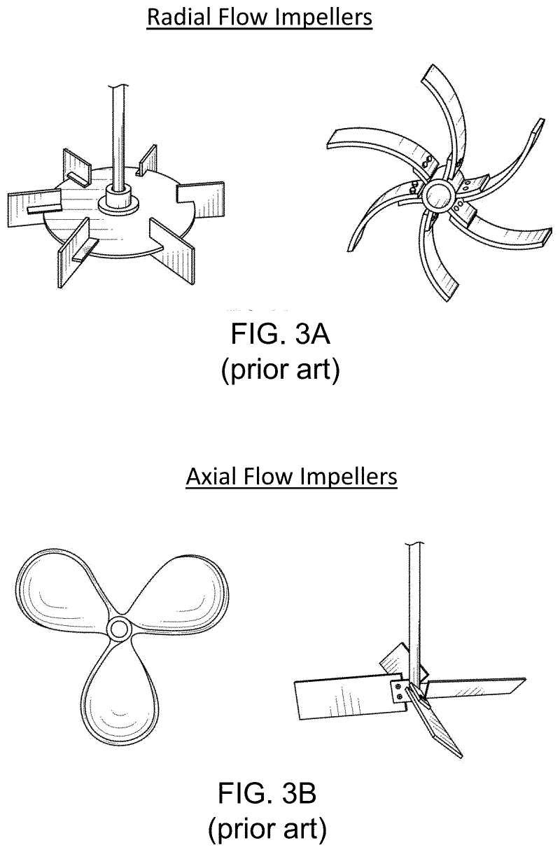

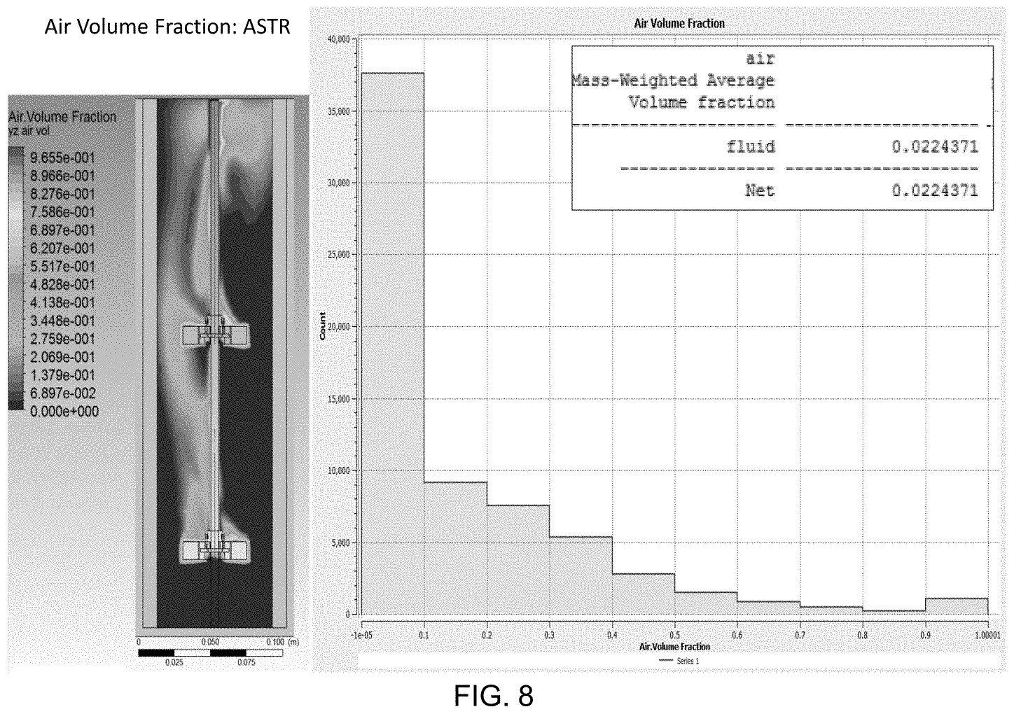

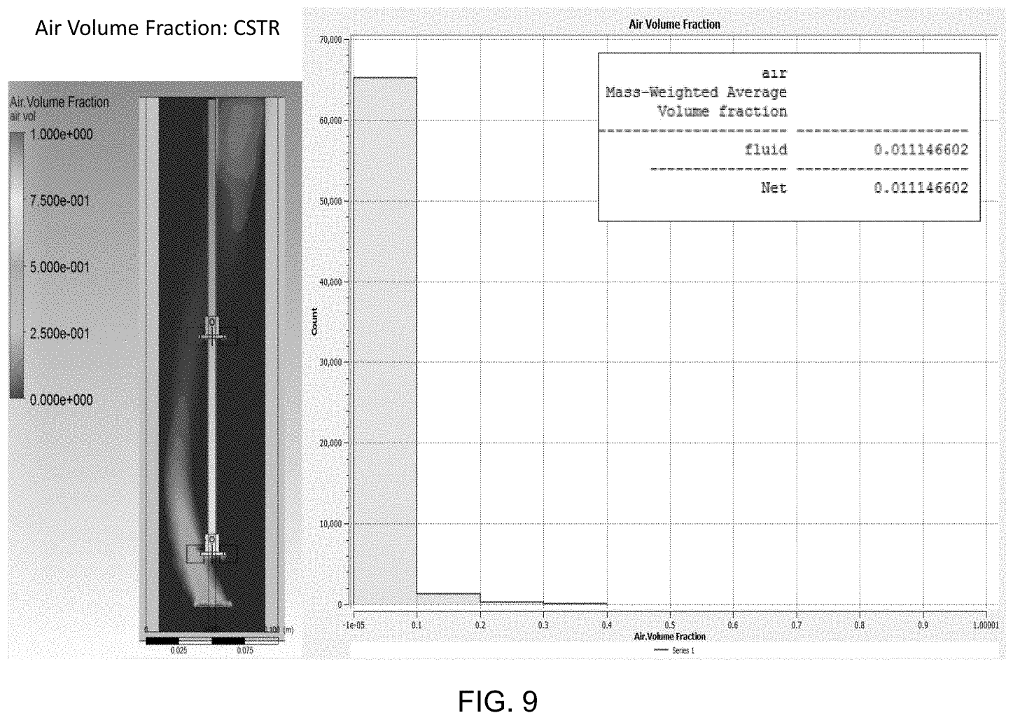

Computational Fluid Dynamics (CFD) simulations were conducted on an Air Stirred Tank Reactor (ASTR), non-limiting examples of an ASTR are shown in FIGS. 1A-1B, and the results from the ASTR simulation are shown in FIGS. 8, 10, and 12. Computational Fluid Dynamics simulations also were conducted on a (control) Continuous Stirred Tank Reactor (CSTR), non-limiting of a CSTR's simple impeller and sparger ring are shown in FIGS. 2A-2B, and the results from the CSTR simulation are shown in FIGS. 9, 11, and 13. The parameters used in the simulations included: Inlet gas flow rate: 0.05 m/s; Impeller Velocity: 100 rpm; Temperature: 25 C; Bubble Diameter: 0.001 m; and Surface Tension: 0.073. A non-limiting example of the dimensions of an ASTR are provided in Table 1.

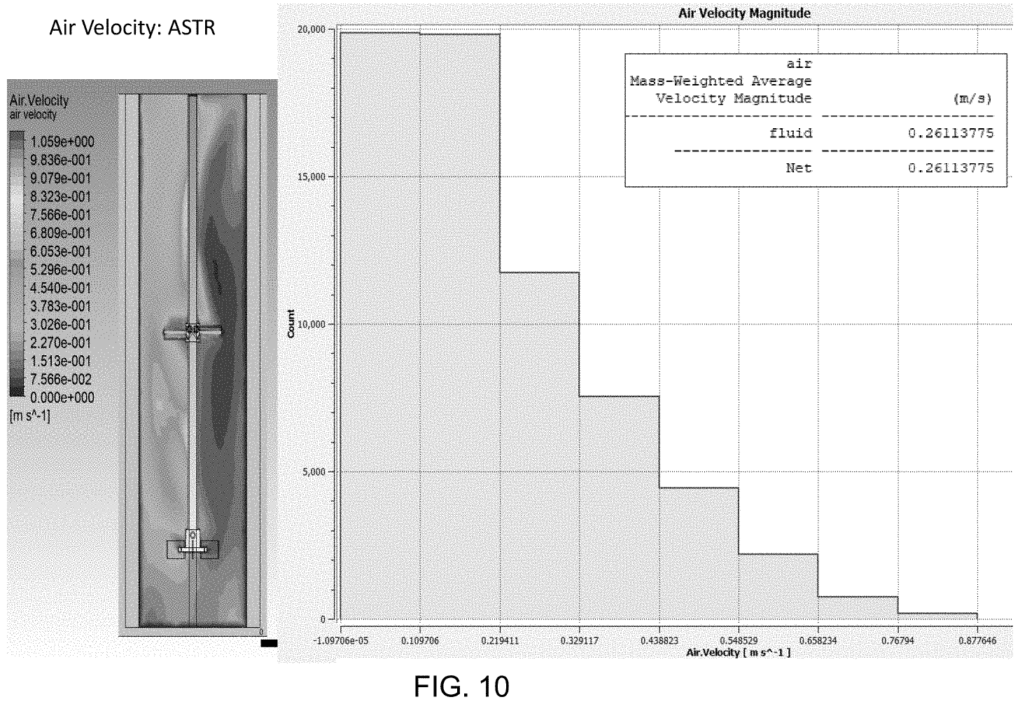

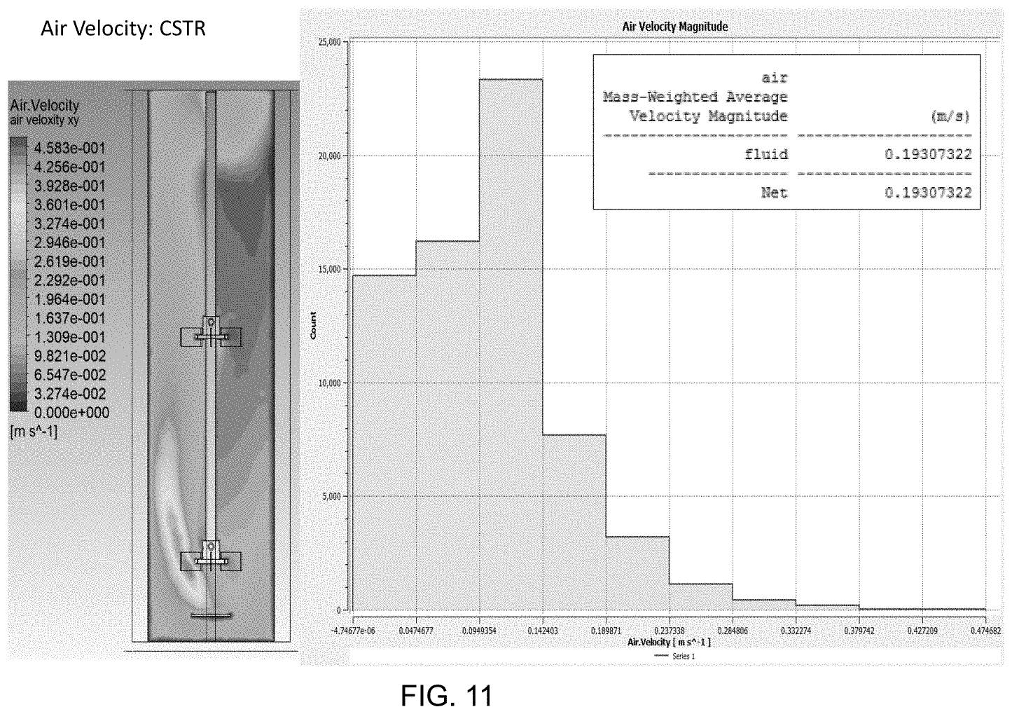

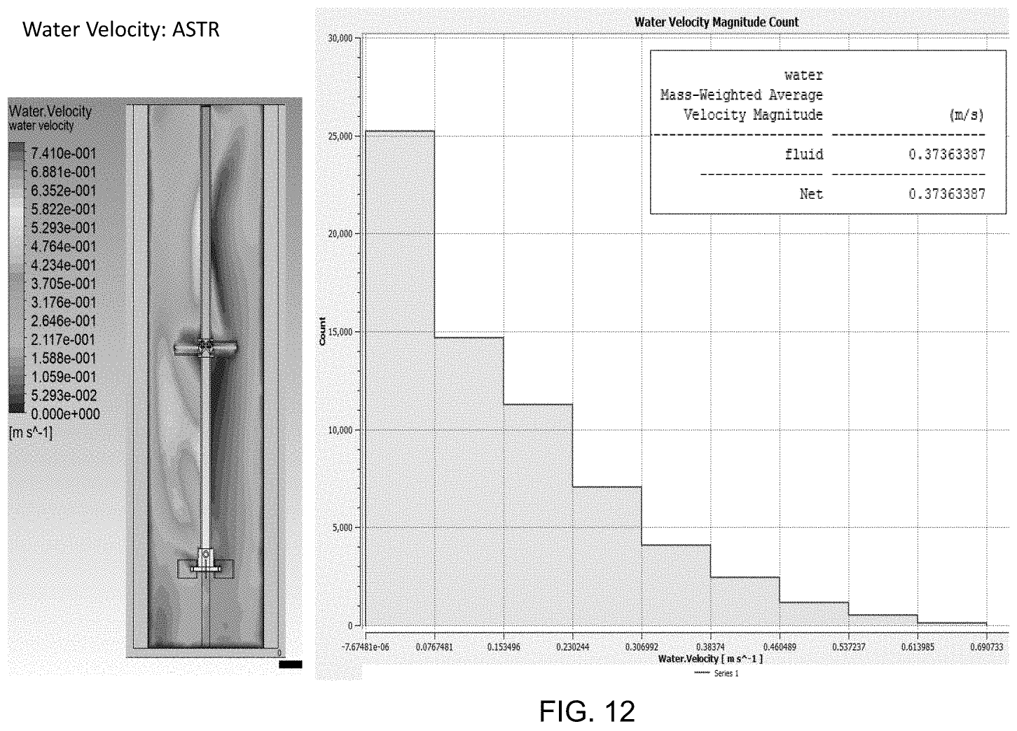

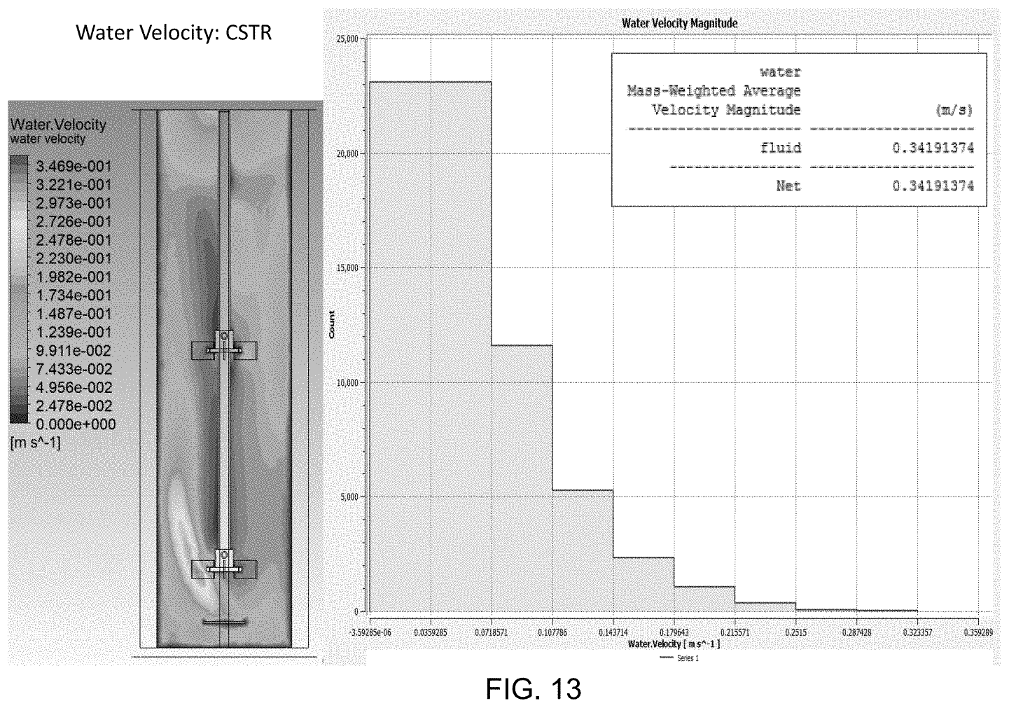

FIGS. 8 and 9, depicting the simulation results for Air Volume Fraction, show that the air mass-weighted average volume fraction in the ASTR exceeded that in the control CSTR by 100%. FIGS. 10 and 11, depicting the simulation results for Air Velocity, show that the air mass-weighted average velocity magnitude in the ASTR exceeded that in the control CSTR by 35%. FIGS. 12 and 13, depicting the simulation results for Water Velocity, show that the water mass-weighted average velocity magnitude in the ASTR exceeded that in the control CSTR by 9%. These results support the significant advantages of the ASTR over the conventional CSTR in terms of gas and liquid mixing as well as gas mass transfer.

TABLE 1
A non-limiting example of the dimensions
of an ASTR of the present invention.

| Reactor Part | Dimension | |
|---|---|---|
| Internal Diameter of | 100.05 | mm |
| Reactor | | |
| External Diameter of | 110.00 | mm |
| Reactor | | |
| External Height | 300.00 | mm |
| Bottom Impeller | 50.00 | mm |
| Clearance | | |
| Distance Between | 100.0 | mm |
| Impellers | | |
| Height of impeller shaft | 250.00 | mm |
| Working Volume | 1.77 | L |
| Baffle Width | 10.00 | mm |

| | |
|---|---|
| Impeller Type | Rushton Turbine |

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the below claims are solely for ease of examination of this patent application, and are exemplary, and are not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A gas-sparging mixing system (110) comprising at least one impeller (120) for circulating fluids, said impeller (120) having pores (125) disposed on a surface (122) of the impeller; and a gas-delivering channel (130) fluidly connected to the impeller (120), wherein the gas-delivering channel (130) is configured to transport gas through the impeller (120), wherein the gas exits through the pores (125) of the impeller, wherein the fluids being circulated by the impeller (120) is sparged with the gas exiting through the pores (125).

2. The system (110) of claim 1, wherein the pores (125) are embedded through the surface (122) of the impeller or embedded on a sparger tape (140) that is attached to the surface (122) of the impeller.

3. The system (110) of claim 1, wherein the pores (125) are embedded on a sparger tape (140) attached to the surface (122) of the impeller.

4. The system (110) of claim 1, wherein the impeller further comprises a light source (150) disposed on the surface (122) of the impeller or embedded within the impeller such that the light source (150) is exposed to the fluids.

5. The system (110) of claim 4, wherein the light source (150) comprises a light emitting diode or light tape.

6. The system (110) of claim 1, wherein the impeller further comprises a light source (150) completely embedded or contained within the impeller such that the light source (150) is not directly in contact with the fluids.

7. The system (110) of claim 1, wherein the gas-delivering channel (130) comprises tubes fluidly connected to the impeller (120) such that gas is transported through the tubes and exits the impeller via the pores (125).

8. The system (110) of claim 1, wherein the impeller (120) is operatively connected to a rotatable shaft (127), wherein the impeller (120) is configured to rotate upon axial rotation of the rotatable shaft (127), wherein the gas-delivering channel (130) comprises tubes fluidly connected to the impeller (120) such that gas is transported through the tubes and exits the impeller via the pores (125), wherein the tubes of the gas-delivering channel (130) is disposed through an interior channel (128) of the rotatable shaft.

9. The system (110) of claim 1, wherein the pores (125) are embedded in a sparger tape (140) attached to the surface (122) of the impeller, wherein the gas-delivering channel (130) comprises tubes fluidly connected to the impeller (120) such that gas is transported through the tubes and exits the impeller via the pores (125), wherein the tubes of the gas-delivering channel (130) are fluidly connected to the sparger tape (140) such that gas is transported through the tubes and exits through the pores (125) of the sparger tape.

10. The system (110) of claim 1, further comprising a plurality of impellers (120) operatively connected to the rotatable shaft (127), wherein the impellers (120) are arranged parallel to each other.

11. The system (110) of claim 1, wherein impeller (120) is a flat blade turbine, a spiral turbine, a propeller, a pitched blade turbine, a helical ribbon impeller, a helical screw impeller, a helical ribbon screw impeller, or an anchor impeller.

12. A method of mixing and sparging gas into a liquid medium, said method comprising:
a) placing a gas-sparging mixing system (110) according to claim 1 in the liquid medium;
b) rotating the impeller (120) to cause circulation of the liquid medium;
c) transporting gas to the impeller (120) via the gas-delivering channel (130), wherein the gas exits through the pores (125) of the impeller; and
d) sparging the liquid medium with the gas while simultaneously mixing the liquid medium.

13. A method of cultivating a liquid culture contained in a reactor vessel (105), said method comprising:
a. providing a gas-sparging mixing system (110) according to claim 1;
b. placing the gas-sparging mixing system (110) in the reactor vessel (105) containing the liquid culture;
c. rotating the impeller (120) to cause circulation of the liquid culture in the reactor vessel (105);
d. transporting gas to the impeller (120) via the gas-delivering channel (130), wherein the gas exits through the pores (125) of the impeller; and
e. sparging the liquid culture with the gas, wherein sparging of the gas further assists in mixing the liquid culture.

14. The method of claim 13, further comprising illuminating the liquid culture using a light source (150).

15. The method of claim 14, wherein the light source (150) is disposed on the surface (122) of the impeller or embedded within the impeller such that the light source (150) is exposed to the liquid culture.

16. The method of claim 14, wherein the light source (150) is completely embedded or contained within the impeller such that the light source (150) is not directly in contact with the liquid culture.

17. An air-stirred tank reactor (ASTR) (100) for cultivation of a liquid culture, said ASTR (100) comprising a reactor vessel (105) configured to contain the liquid culture, and a gas-sparging mixing system (110) according to claim 1 disposed in the reactor vessel (105), wherein the impeller (120) of the gas-sparging mixing system is configured to circulate the liquid culture, wherein the gas-delivering channel (130) is configured to transport gas through the impeller (120), which exits through the pores (125) of the impeller, wherein the liquid culture being circulated by the impeller (120) is sparged with the gas exiting through the pores (125), thereby creating a synergistic effect of fluid mixing and gas sparging in the liquid culture.

18. The ASTR (100) of claim 17, wherein the impeller (120) further comprises a light source (150) disposed on the surface (122) of the impeller or embedded within the impeller such that the light source (150) is exposed to the fluids, wherein the light source (150) provides lighting inside the reactor vessel (105).

19. The ASTR (100) of claim 17, wherein the light source (150) comprises a light emitting diode or light tape.

20. The ASTR (100) of claim 17, wherein the impeller further comprises a light source (150) completely embedded or contained within the impeller such that the light source (150) is not directly in contact with the fluids, wherein the light source (150) provides lighting inside the reactor vessel (105).
